(19) United States
(12) Patent Application Publication
(10) Pub. No.: US 2015/0245925 A1
(43) Pub. Date: Sep. 3, 2015

(54) CUTTING ASSEMBLY AND METHOD OF USE

(71) Applicant: BACTERIN INTERNATIONAL, INC., BELGRADE, MT (US)

(72) Inventors: Joel Scott Willyerd, Belgrade, MT (US); Todd R. Meyer, Bozeman, MT (US); Craig S. Smith, Bozeman, MT (US); Dustin B. Byrd, Belgrade, MT (US); Helena M. Lovick, Belgrade, MT (US); Daniel Cox, Bozeman, MT (US); Michael Mansfield, Bozeman, MT (US)

(21) Appl. No.: 14/634,577

(22) Filed: Feb. 27, 2015

Related U.S. Application Data
(60) Provisional application No. 61/946,543, filed on Feb. 28, 2014.

(51) Int. Cl. A61F 2/46; B02C 18/22; B02C 18/18

(52) U.S. Cl. CPC A61F 2/4644; B02C 18/186; B02C 18/2233; A61F 2002/4645

(57) ABSTRACT

The invention relates to a cutting assembly for preparing fibers and methods for using the same. The invention may produce a plurality of fibers of different shapes and lengths, which may depend upon the operating parameters.

### CROSS REFERENCE TO RELATED APPLICATIONS

This application claims priority under 35 U.S.C. §119(e) to U.S. Provisional Patent Application Ser. No. 61/946,543, filed on Feb. 28, 2014, which is incorporated herein in its entirety by reference.

### FIELD OF THE INVENTION

The present embodiments generally relate to a cutting assembly for producing shredded fibers from a solid material.

### BACKGROUND

Machines for shredding various materials are known in the prior art. The majority of these machines and cutting assemblies provide a shredded product of uncontrolled size. The cutting assemblies that provide fibers of controlled sizes fail to allow sufficient user control over the size and shape of the resultant fibers. A need remains for a cutting assembly capable of producing fibers of adjustable and controllable sizes. It is desirable for a cutting assembly to provide user adjustment of the cutting of fibers to accommodate different material types, physical properties, and end product specifications. A need also remains for a cutting assembly capable of user adjustment of the cutting direction of the assembly with respect to the material being cut. Additionally, it is desirable for a cutting assembly capable of user adjustments to material cutting direction and high fiber yields without burdensome limitations due to material clamping requirements, e.g., the need to grip the material being cut.

U.S. Pat. No. 7,582,309 entitled “Cohesive Demineralized Bone Compositions” discloses the use of DBM composition of fibers with lengths between 250 μm to 2 mm and a biocompatible liquid to form a coherent, formable mass. The fibers are formed when bone is shaved. U.S. Pat. No. 3,856,219 entitled “Bone Mill” discloses a mill for converting bone into controlled size fragments. A cutter bar shears the bone into fragments and is air driven. The mill is also autoclavable. U.S. Pat. No. 6,318,651 entitled “Mill, In Particular for Milling of Bone, as well as a Drum, Provided with Cutting Members, Applicable in the Mill” discloses a mill for bone with a drum provided. The cutting members are rotatably located inside the housing. The cutting members on the drum are characterized in that the cutting members are formed by local thickenings of the wall of the drum. U.S. Publication No. 2011/0172671 entitled “Orthopedic/Spine Bone Mill” discloses a bone mill comprising a vessel and a rotatable grinding tool. The vessel further includes a generally concave or a cup shaped bottom wall to correspond to the shape of the cutting blade and lid to be removably connected to the vessel. The grinding tool includes an elongated shaft extending through the opening in the lid with a cutting blade mounted to the shaft which may be rotated by a device such as a drill.

### SUMMARY OF THE INVENTION

The disclosed invention is directed to a cutting assembly capable of producing fibers of adjustable and controllable sizes based on a user's requirements. The cutting assembly of this invention includes a holding structure, a rotating, cylindrical cutting shaft or drum, and a feed plate to hold the material to be cut against the cutting shaft. Additional optional features of the cutting assembly are also disclosed. Optional features include windows for visibility, exit ports, collection trays at the exit ports, and so forth.

In an embodiment, the cutting shaft of the assembly may be interchanged with different cutting shafts to adjust the cutting surface to suit the type of material to be cut. This adjustability allows for more user control over the sizing of fibers produced by the cutting assembly.

To use the cutting assembly of this invention, the cutting shaft is mounted into the housing structure. A motor or hand crank via a gearbox or other power source rotates the cutting shaft. The feed plate of the assembly is pressed against the article to be cut forcing the article into contact with the cutting shaft and holding it in place. As the cutting shaft rotates, the shaft cuts the material into fibers. The size and shape of the fibers may be further controlled by the orientation of the article being cut (e.g., perpendicular or parallel to the cutting surface, or at any other angle between about zero degrees and about 90 degrees), amount of contact of the article to be cut against the cutting shaft, adjustments to the surface of the cutting shaft, rotation speed of the cutting shaft, and feed pressure of the plate against the material to be cut.

### BRIEF DESCRIPTION OF THE FIGURES

FIG. 1 illustrates a perspective, isometric view of the cutting assembly of the present invention; and

FIG. 2 illustrates a perspective, top view of the cutting assembly.

### DETAILED DESCRIPTION OF THE INVENTION

An aspect of the invention is a cutting assembly for preparing a plurality of fibers. The cutting assembly includes a holding structure, a rotating cutting shaft, and a feed plate. The feed plate presses the article to be cut against the rotating cutting shaft.

The material of the rotating cutting shaft may be ceramic, stainless steel, brass, aluminum, carbon steel, or combinations thereof. In some embodiments, the rotating cutting shaft may be interchangeable, or may be fitted with cutting blades at a point of contact that contains the article. In some embodiments, the rotating cutting shaft may be coated with a protective coating that may be chromate, anodize, electroplate, galvanization metals or the like.

The mechanism may rotate the cutting shaft at variable speeds, which may be adjusted by the user. The variable speed of the cutting shaft facilitates the production of the plurality of fibers, may result in different shapes and lengths.

The article to be cut may be held in place by a force of the feed plate pressed against the article or attached to the feed plate. The feed plate may be adjustable to accommodate multiple sizes of articles. The article may be moved against the cutting shaft at an angle, and the angle may be between about zero to about 90 degrees. Thus, the article may be moved perpendicular to the cutting shaft or parallel to the cutting shaft. For example, if the article is a material that is elongated, such as a piece of bone, then the elongated axis of the bone may be perpendicular or parallel to the cutting shaft.

The holding structure may further include at least one exit port that may be adjacent or underneath the holding structure. The fibers may be collected in at least one collection tray, which may be attached to the assembly. The tray may be underneath the assembly or adjacent to the assembly. In some embodiments, the holding structure may include a cover, and the cover may be impact resistant. The holding structure may be fully or partially transparent. In some embodiments, more than about 90% of the holding structure may be transparent. In some embodiments, the holding structure may contain at least one impact-resistant window, which may be transparent.

An aspect of the invention is a method of shredding an article into a plurality of fibers using a cutting assembly. The method includes placing the article against a feed plate, rotating the cutting shaft, and placing the article against the cutting shaft to produce fibers.

The material of the article to be cut may be foodstuffs, wood, elastomers, thermoplastics, metal, bone, or combinations thereof. The article may be attached to the feed plate or held against the cutting shaft by a force of the feed plate pressed against the article. The article may be moved against the cutting shaft at an angle, which may be between about zero to about 90 degrees. Thus, the article may be cut perpendicular to the cutting shaft or parallel to the cutting shaft.

In some embodiments, the length of the plurality of fibers may be between about 1 mm and about 200 mm. A diameter of the plurality of fibers may be between about 0.1 mm and about 30 mm.

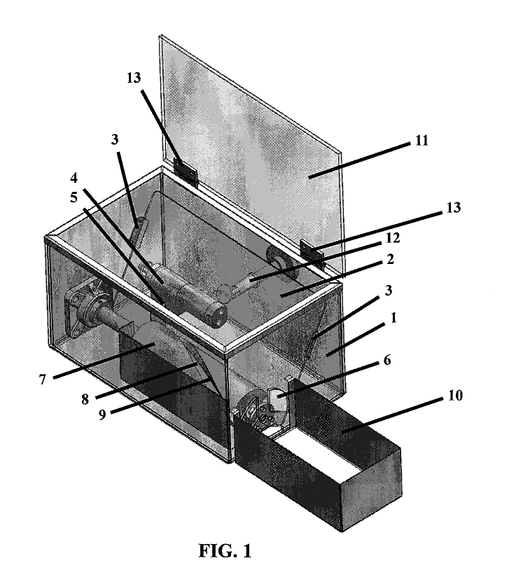

As illustrated in FIG. 1, the cutting assembly of the present invention includes a holding structure 1, a rotating, cylindrical cutting shaft 7, and a feed plate 2 to hold the material to be cut against the cutting shaft. The holding structure 1 for the cutting assembly may be composed of various materials including metal, or plastic or combinations thereof. The holding structure 1 may be transparent as shown, or contain at least one viewing window to allow visualization of the cutting process. A power source, such as a motor 4, or hand crank via a gearbox 5 may rotate the cutting shaft.

The feed plate 2 may be attached to the holding structure with an attachment piece 3, for example through and with brackets or hinges. The attachment piece 3 may allow adjustment of the space between the feed plate 2 and the cutting shaft 7. In some embodiments, the feed plate 2 may be removable. In further embodiments, the feed plate 2 may be exchangeable with other feed plates. This exchangeability of the feed plates would allow user selection of different feed plate features such as attachment features matched to the article to be cut.

In further embodiments, a pressure adjustment rod 12 may be attached to the feed plate 2. The pressure adjustment rod 12 may be manual or motor-driven. The pressure adjustment rod 12 may allow a pre-established stop to prevent contact of the feed plate 2 and/or any clamping features with the cutting shaft 7. The adjustable features of the feed plate 2 may allow variable pressure to be placed against the material being cut, which may accommodate different material types and sizes. In some embodiments, the article to be cut may be held in place by the force of the feed plate 2 pressing the article onto the cutting shaft 7 surface. In other embodiments, the feed plate 2 may allow attachment of the article to be cut to the feed plate. The attachment of the article to be cut onto the feed plate 2 may allow the article to be moved against the cutting shaft 7 in a motion parallel to the cutting shaft 7. In other embodiments, the attachment of the article to be cut onto the feed plate 2 may allow the article to be moved against the cutting shaft 7 in a motion perpendicular to the cutting shaft 7. In further embodiments, the attachment of the article to be cut onto the feed plate 2 may allow the article to be orientated at various angles between about zero to about 90 degrees against the cutting shaft 7. Methods of attachments of the article to be cut to the feed plate 2 may include, by way of example only, gripping ridges, spikes, frictional attachments or the like.

In other embodiments, the contact surface area between the article to be cut and the cutting shaft 7 may be maximized by the feed plate 2 via pressure or via attachment of the article to be cut to the feed plate 2. In other embodiments, the contact surface area between the article to be cut and the cutting shaft 7 may be minimized by the feed plate 2, for example via pressure or via attachment of the article to be cut to the feed plate 2. The ability to minimize or maximize the contact surface area may allow adjustment of the resultant fiber sizes and shapes. The holding structure 1 may include a cover or lid 11 to prevent undesired displacement of the article to be cut and as a protective shield for the user. In some embodiments, the lid 11 may feature a safety closure mechanism that prevents the cutting shaft 7 from rotating when the lid 11 is in an open position.

In some embodiments, a feeding funnel or apparatus may be attached to the top of the cutting assembly. A protective cover or lid 11 may be included with the feeding apparatus. The protective cover or lid may be impact resistant.

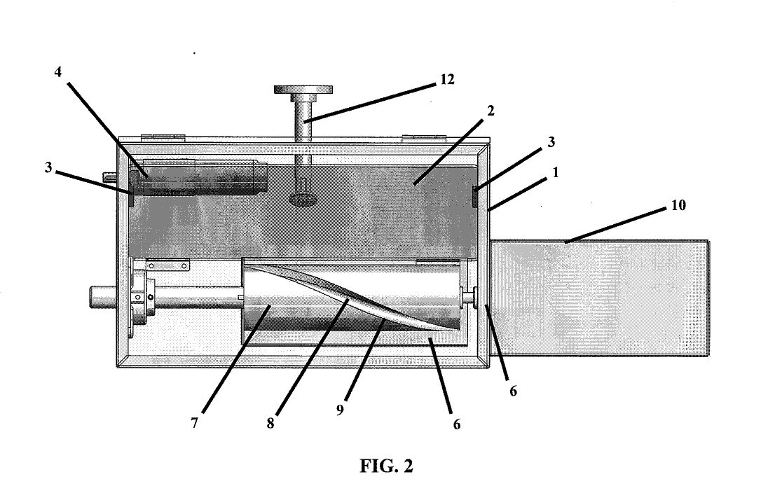

As illustrated in FIG. 2, the holding structure 1 may contain one or more exit ports 6 to allow removal of the cut material. The exit port 6 may be perpendicular to the feed plate 2 and/or underneath the holding structure, opposite the material input side of the holding structure 1. At least one collection tray 10 may be attached or next to the holding structure to at least one exit port in order to collect the cut material as it is produced. In a preferred embodiment the collection tray or trays 10 may be emptied while the cutting assembly is in use, to remove excess material and prevent clogging of the assembly. As an additional feature, a grid or screen may be placed in the exit port 6 to slow the exit of material from the cutting assembly. The grid or screen may also serve as a size barrier to ensure additional cutting of the article until the cut pieces are sufficiently reduced in size to exit from the assembly. In some embodiments, the grid or screen within the exit port may be removable or interchangeable with other grid structures to allow adjustments for different materials to be cut and for additional sizing cut options.

As illustrated in FIG. 2, the cutting shaft 7 may consist of at least one flute 8 to provide the desired amount of cutting edges 9. In some embodiments, between 1 to 10 flutes may be on the cutting shaft. The helical angle of the flutes, the flutes width, blade angle, and blade depth may be adjusted for the specific cutting application. By way of non-limiting example, the helical angle may be between about 0 to about 90 degrees, in some embodiments, between about 80 degrees to about 90 degrees. By way of non-limiting example, the flute width may be between about 1/16^{th} of an inch to about 0.5 inches, in some embodiments about a quarter of an inch. The blade angle with respect to the article to be cut (for example) may be between about 0 degrees to about 90 degrees. In some embodiments, the blade angle may be about zero degrees. By way of non-limiting example, the blade depth may be between about 1/16^{th} of an inch to about 0.5 inches. In some embodiments, the blade depth may be about a quarter of an inch. The cutting shaft 7 may be composed of ceramic, stainless steel, brass, aluminum, carbon steel, other metal alloys, or combinations thereof.

In some embodiments, the cutting shaft 7 may be coated with a protective covering such as carbide, chromate, anodize, electroplate, galvanization metals and the like. In other embodiments, the cutting shaft 7 may be fitted with cutting blades at the point of contact with the fibers. The cutting shaft 7 may be removable from the holding structure. This removability would allow replacement of a worn shaft, facilitate cleaning, and allow selection of different types of cutting shafts based upon the user's needs.

The cutting assembly may be equipped with a mechanism that allows variable rotation of the cutting shaft 7. This mechanism may be part of the power source 4, gearbox 5, or a separate piece of equipment. Variable rotation of the cutting shaft 7 allows the user to adjust the cutting speed for differences in the materials being cut, such as densities, size, and material type. The power source 4 for rotating the cutting shaft 7 may be pneumatic or electric. In another embodiment, the cutting shaft 7 may be powered manually, such as via a hand crank.

In an embodiment, the cutting assembly may be composed of heat stable materials to allow steam sterilization in whole or in part of the assembly. Another embodiment of the present disclosure is a method of shredding material into a plurality of fibers using the cutting assembly. The materials to be cut may consist of foodstuffs, wood, elastomers, thermoplastics, metal, bone or combinations thereof. The type of bone may be cortical, cancellous, or a combination of bone types. The bone may be mineralized, fully demineralized, or partially demineralized. The materials cut using this method may provide fiber lengths of between about 1 mm to about 200 mm, about 2 mm to about 150 mm, about 5 mm to about 70 mm, to about 10 mm to about 60 mm. The average length of the fibers may be between about 15 mm to about 50 mm, in some embodiments about 30 mm. The materials cut using this method may provide fiber with a width or diameter between about 0.1 mm to about 30 mm, about 0.2 mm to about 15 mm, about 0.5 mm to about 10 mm, to about 1 mm to about 8 mm. The average width or diameter may be between about 1 mm to about 5 mm, in some embodiments between about 2-3 mm. Additional user control over the shape and size of the plurality of fibers generated using the cutting assembly of the present invention may be provided in embodiments where the article to be cut may be attached to the feed plate 2 and moved in a motion perpendicular, parallel, or various angles (e.g., about 0° to about 90°) to the cutting shaft 7.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A cutting assembly for controlled generation of a plurality of fibers, comprising:
a holding structure;
a rotatable cutting shaft; and
a feed plate to press an article to be cut against the rotatable cutting shaft.

2. The cutting assembly of claim 1, wherein the rotatable cutting shaft is interchangeable.

3. The cutting assembly of claim 1, wherein a material of the rotatable cutting shaft is selected from the group consisting of a ceramic, a stainless steel, a brass, an aluminum, a carbon steel, and combinations thereof.

4. The cutting assembly of claim 1, wherein the rotatable cutting shaft is fitted with at least one cutting blade at a point of contact, and wherein the point of contact indicates an angle to the article with the at least one cutting blade.

5. The cutting assembly of claim 1, wherein the rotatable cutting shaft is coated with at least one protective coating.

6. The cutting assembly of claim 5, wherein the at least one protective coating is selected from the group consisting of chromate, anodize, electroplate, galvanization metals and combinations thereof

7. The cutting assembly of claim 1, wherein a mechanism rotates the cutting shaft at a variable speed.

8. The cutting assembly of claim 7, wherein the variable speed of the cutting shaft facilitates the plurality of fibers of a particular shape.

9. The cutting assembly of claim 1, wherein the article to be cut is held in place by a force of a feed plate pressed against the article.

10. The cutting assembly of claim 1, wherein an article to be cut is attached to the feed plate.

11. The cutting assembly of claim 10, wherein the article is moved against the cutting shaft at an angle.

12. The cutting assembly of claim 11, wherein the angle is between about zero to about 90 degrees.

13. The cutting assembly of claim 10, wherein the article is moved against the cutting shaft in a parallel direction.

14. The cutting assembly of claim 10, wherein the article is moved against the cutting shaft in a perpendicular direction.

15. The cutting assembly of claim 1, wherein the holding structure comprises at least one exit port.

16. The cutting assembly of claim 1, wherein the plurality of fibers are collected in at least one collection tray.

17. The cutting assembly of claim 1, wherein the rotatable cutting shaft comprises a helical flute with at least one cutting edge.

18. The cutting assembly of claim 1, wherein the holding structure comprises an impact resistant cover.

19. The cutting assembly of claim 1, wherein more than about 90% of the holding structure is transparent.

20. The cutting assembly of claim 1, wherein the holding structure contains at least one transparent impact-resistant window.

21. The cutting assembly of claim 1, wherein the feed plate is adjustable to accommodate multiples sizes of the articles.

22. The cutting assembly of claim 1, wherein a cutting speed of the assembly is adjustable.

23. A method of shredding an article into a plurality of fibers using a cutting assembly, comprising:
placing the article against a feed plate;
rotating a cutting shaft; and
placing the article against the cutting shaft.

24. The method of claim 27, wherein a material of the article to be cut is selected from the group consisting of foodstuffs, wood, elastomers, thermoplastics, metal, bone, and combinations thereof

25. The method of claim 27, wherein the article is attached to a feed plate.

26. The method of claim 27, wherein the article is held against the cutting shaft by a force of a feed plate pressed against the article.

27. The method claim 27, wherein the article is moved against the cutting shaft at an angle between about zero to about 90 degrees.

28. The method of claim 27, wherein the article is cut perpendicular to the cutting shaft.

29. The method of claim 27, wherein the article is cut parallel to the cutting shaft.

30. The method of claim 28, wherein the material is bone.

31. The method of claim 32, wherein the bone is cortical bone.

32. The method of claim 27, wherein a length of the plurality of fibers are between about 1 mm and about 200 mm.

33. The method of claim 27, wherein a diameter of the plurality of fibers are between about 0.1 mm and about 30 mm.

34. The cutting assembly of claim 9, wherein the article is moved against the cutting shaft at an angle.

35. The cutting assembly of claim 9, wherein the article is moved against the cutting shaft in a parallel direction.

36. The cutting assembly of claim 9, wherein the article is moved against the cutting shaft in a perpendicular direction.

37. The cutting assembly of claim 7, wherein the variable speed of the cutting shaft facilitates the plurality of fibers of a length.
